(19) United States
(12) Patent Application Publication
(10) Pub. No.: US 2010/0256560 A1
(43) Pub. Date: Oct. 7, 2010

(54) DISPOSABLE SAFETY SYRINGE WITH AN AUTOMATICALLY RETRACTABLE NEEDLE

(76) Inventor: Cherry LI, Zhejiang Province (CN)

Correspondence Address: OSTROLENK FABER GERB & SOFFEN, 1180 AVENUE OF THE AMERICAS, NEW YORK, NY, 100368403, US

(21) Appl. No.: 12/569,156

(22) Filed: Sep. 29, 2009

(30) Foreign Application Priority Data
Apr. 4, 2009 (CN) 200920116899.8

(51) Int. Cl. A61M 5/50

(52) U.S. Cl. 604/110

(57) ABSTRACT

A disposable safety syringe with an automatically retractable needle is provided, which comprises a cylinder, a plunger and a needle seat. The needle seat is comprised of a large needle seat and a small needle seat, in which the large needle seat is connected with the cylinder. The small needle seat is disposed within the large needle seat for mounting the needle. The interior of the plunger is a through hole and an elastically reset device is provided in the front portion of the through hole for taking the small needle seat back into the through hole of the plunger after injection.

The safety syringe according to the present invention is reasonable and simple in structure, easy in processing, low in cost and is superior and reliable in performance.

### TECHNICAL FIELD

The present invention generally relates to a medical article, particularly to a disposable safety syringe with an automatically retractable needle.

### BACKGROUND

Syringe is a common medical instrument. Currently all the syringes widely used by medical institutions are disposable articles. This kind of theoretically “disposable” syringes still has a risk to be reused by lawbreakers and have a safety problem that the needle is exposed during the wastes process. Statistics from the Mainland of China show that cross infection and malignant transmission of diseases due to reuse of a disposable syringe, needle sharing and mishandling of wastes have already caused 390 thousand of countrymen to die earlier and resulted in loss of 6.89 million life years with 142 million US dollars of direct medical expenses.

In addition, the disposable syringes present in the domestic market cannot ensure implementation of safe injection because they have shortages in structure and technical solution. Due to the use of the unsafe self-destruction needling instruments, needle injuries to the medical staff are obviously more than damages caused by other accidences. Based on statistics, the domestic clinical injections are about 13 billion times/year, while unsafe injections take more than 60 percent thereof. In addition, according to the statistics by experts under the State Ministry of Health, about 80 percent of infections of the hematogenic transmitted diseases, such as hepatitis B, hepatitis C and AIDS, are caused by needle stabs. This will inevitably cause second cross infection of pathogenic bacteria, which bring about serious threat to the personal safety of the medical staff.

Ye Lei, Chinese director of Global AIDS Project of the U.S. Center for Disease Control and Prevention, pointed out that the best way to solve unsafe injection is to use disposable self-destruction syringes. In 1994, WHO (World Health Organization) raised requirements and definition of safe injection: no harm to the receiver, no harm to the user, and no harm to the environment.

### SUMMARY

Aiming at the foregoing problem, the object of the present invention is to provide a disposable safety syringe with an automatically retractable needle, which is reasonable and simple in structure, easy in processing, low in cost and superior and reliable in performance.

The foregoing object is achieved by the following technical solution.

A disposable safety syringe with an automatically retractable needle, comprising a cylinder, a plunger and a needle seat, characterized in that: the needle seat is comprised of a large needle seat and a small needle seat, in which the large needle seat is connected with the cylinder; the small needle seat is disposed within the large needle seat for mounting the needle; the interior of the plunger is a through hole and an elastically reset device is provided in the front portion of the through hole for taking the small needle seat back into the through hole of the plunger after injection. The small needle seat is fitted within the large needle seat by virtue of a static friction force.

Further, small needle seat is provided with an inner hole in the center thereof and the rear portion of the inner hole is a connection hole section, a snap-fit projection being provided at the bottom of the connection hole section to cooperate with the elastically reset device for position restriction.

Further, the front portion of the inner hole of the small needle seat is a fine through hole with a needle mounted thereon, the rear portion of the inner hole of the small needle seat is comprised of a transition hole section and a connection hole section with the inner diameter of the transition hole section smaller than that of the connection hole section, and the connection hole section being provided with a snap-fit projection at the bottom thereof; the interior of the plunger is a through hole, the through hole being provided at the front portion thereof with an elastically reset device to take the small needle seat back into the through hole of the plunger after injection, the through hole is hermetically engaged with a push handle at the tail portion thereof.

Further, the external bottom of the large needle seat is provided with a convex ring, the head portion of the cylinder is provided with a recess with a thread provided therein to connect with the convex ring.

Further, the external middle of the large needle seat is provided with a ratchet to engage with a sheath.

Further, the inner hole of the large needle seat is comprised of an upper hole section, an intermediate hole section and a lower hole section with their hole diameters successively increasing from the top to the bottom, the upper hole section and the intermediate hole section defining an upper flange therebetween, and the intermediate hole section and the lower hole section defining a lower flange therebetween; the small needle seat is comprised of an upper section, an intermediate section and a lower section with their hole diameters successively increasing, the upper section and the intermediate section defining an upper boss therebetween, and the intermediate section and the lower section defining a lower boss therebetween; the upper section is located within the upper hole section of the large needle seat, the intermediate section being located within the intermediate hole section of the large needle seat and being hermetically fit therewith, and the lower section being fitted with the mounting hole of the cylinder; and the top of the cylinder and the upper boss of the small needle seat abut against the upper flange of the large needle seat, and the lower boss of the small needle seat abutting against the lower flange of the large needle seat.

Further, a stop structure is provided between the intermediate section of the small needle seat and the intermediate hole section of the inner hole of the large needle seat to prevent the small needle seat from rotating, which comprises a lug disposed on the intermediate section of the small needle seat and a depression disposed on the intermediate hole section of the large needle seat.

Preferably, the length of the connection hole section of the inner hole of the small needle seat is on the order of 1.5 to 2 mm.

Further, the through hole is provided therein with a boss surface to divide the inner hole into a small hole diameter section and a large hole diameter section, and the opening portion of the small hole diameter section being provided with the friction flange.

Further, the elastically reset device is comprised of a spring fixed seat and a compression spring; in which under the action of the compression spring, the spring fixed seat forms with the through hole of the plunger a movable fit that has temporary restraint, and in which the spring fixed seat comprises a head portion and an upper trunk portion protruding out of the through hole, and a lower trunk portion and a foot portion within the through hole, the top of the head portion of the spring fixed seat being a flat square structure, two sides of the flat square structure being conical bevels, the bottom of the conical bevel protuberating outward to form a snap ring, the maximum outer diameter of the snap ring being smaller than the inner diameter of the connection hole section of the small needle seat but greater than the minimum inner diameter of the snap-fit projection, the snap ring being able to engage with the snap-fit projection of the small needle seat when the spring fixed seat is pulled down; the upper trunk portion has a length smaller than that of the connection hole section of the small needle seat and has an outer diameter smaller than the minimum inner diameter of the friction flange but greater than that of an mounting hole at the head portion of the cylinder; the lower trunk portion is in clearance fit with the smaller hole diameter section of the through hole of the plunger while in tight fit with the friction flange of the through hole; a top end face of the foot portion abuts against the boss surface of the through hole, the foot portion protuberating outward at the bottom thereof to form a block, the compression spring being mounted between the block and the boss surface of the through hole, the restoring force of the compression spring is smaller than the friction generated by the interference fit between the lower trunk portion of the spring fixed seat and the friction flange before the compression spring is triggered, and the restoring force can draw the small needle seat and the injection needle back into the plunger after the compression spring is triggered.

Further, the flat square structure on the head portion of the spring fixed seat is provided with a through hole for increasing deformation.

Further, the inner hole of the cylinder is provided with a snap ring at the bottom thereof. (The function of the snap ring: the plunger of the present invention is locked by the snap ring of the cylinder after use and thus cannot be pulled out of the cylinder again. Hence, it can avoid risk of illegal reprocess and reuse.)

Further, the cylinder is provided with a mounting hole at the front portion thereof, and the mounting hole is in connection with the inner hole of the cylinder by a transition bevel.

The present invention has the following advantages over the prior art:

1. The needle of the present invention and its needle seat are always kept within the plunger after the injection and will not protrude out of the front end face of the cylinder to cause accidental injury, thus thoroughly eliminating risk to the operator during the recovery process of syringes.

2. The plunger of the present invention is locked by the snap ring of the cylinder after the injection so as not to be pulled out of the cylinder again, thus avoiding risk of illegal reprocess and reuse.

3. The reasonable and simple structure of the present invention ensures little medicament residual, thus avoiding pollution of the residual medicament liquor to the personnel.

4. The present invention is low in cost, superior, safe and reliable in performance, and can realize a mass production.

5. The needle seat of the present invention is composed of a large needle seat and a small needle seat which are fitted to each other, and the large needle seat is in threaded connection with the cylinder such that the needle and its needle seat can be replaced integrally as required. Thus, it can meet the requirement of drug dispensing and injection under which various types of injection needles are required.

### DESCRIPTION OF FIGURES

FIG. 1 is a structural drawing of an integral assembly of the present invention;

FIG. 2 is a structural drawing of the cylinder of the present invention;

FIG. 3 is a structural drawing of the large needle seat of the present invention;

FIG. 4 is structural drawing of the small needle seat with a needle of the present invention;

FIG. 5 is a structural drawing of the plunger of the present invention;

FIG. 6 is a structural drawing of the spring fixed seat of the present invention;

FIG. 7 is a structural drawing of the of present invention before injection;

FIG. 8 is a partial enlarged drawing of section A in FIG. 7;

FIG. 9 is a structural drawing of the elastically reset device of the present invention before triggered while during injection;

FIG. 10 is a partial enlarged drawing of section B in FIG. 9;

FIG. 11 is structural drawing of the elastically reset device of the present invention after triggered during injection;

FIG. 12 is a partial enlarged drawing of section C in FIG. 11;

FIG. 13 is a partial enlarged drawing of section D in FIG. 11; and

FIG. 14 is a partial drawing of the cylinder mounting hole with a small needle seat stop structure at the bottom thereof according to another embodiment of the present invention.

### EMBODIMENTS

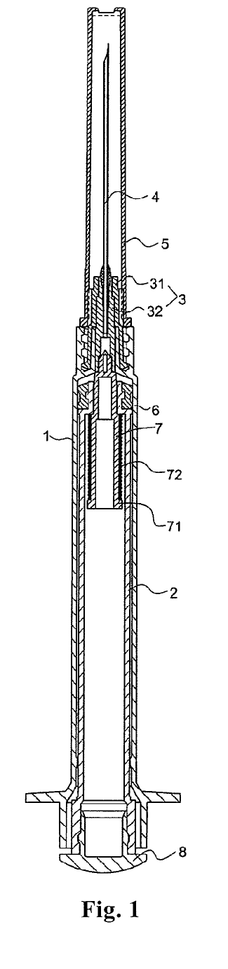

As shown in FIG. 1, a disposable safety syringe with an automatically retractable needle comprises a hollow cylinder 1, a plunger 2 mounted within the cylinder 1, a needle seat 3 mounted at the head portion of the cylinder, and a sheath 5 encased over the needle seat for protecting a needle 4.

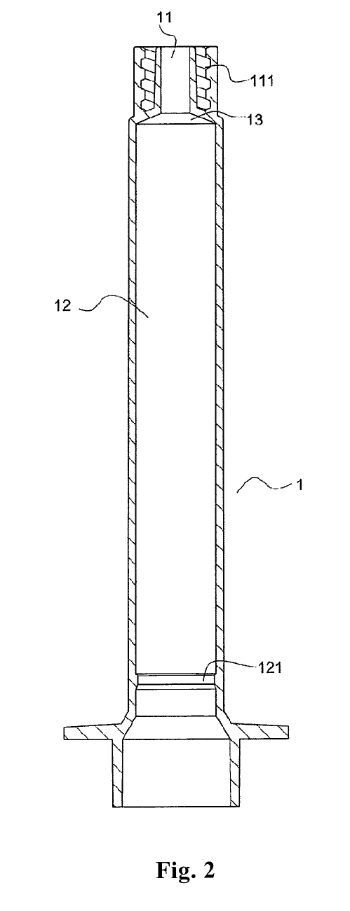

As shown in FIG. 2, the cylinder 1 is provided with a mounting hole 11 at the front portion thereof. The front end face of the cylinder is provided with a recess 111 in which connection threads are provided. The mounting hole 11 is connected with an inner hole 12 of the cylinder by means of a transition bevel 13. The inner hole 12 of the cylinder is provided with a snap ring 121 at the bottom thereof such that the plunger 2 is locked by the cylinder 1 after injection is accomplished and cannot be pulled out of the cylinder 1 again, thus avoiding risk of illegal reprocess and reuse.

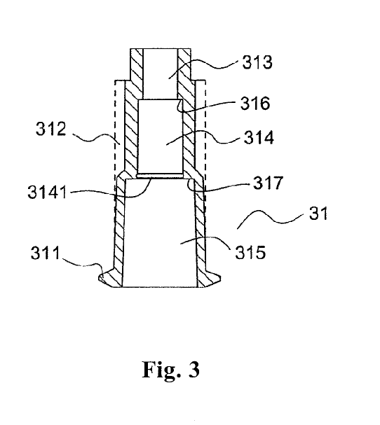

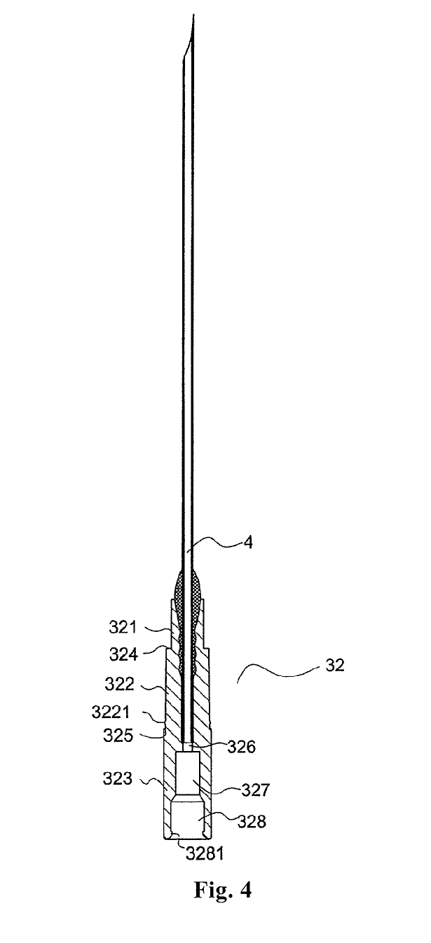

As shown in FIGS. 1 and 3, the needle seat 3 is comprised of a large needle seat 31 and a small needle seat 32, wherein a convex ring 311 is provided at the exterior bottom of the large needle seat 31 to form a threaded connection with the recess 111 provided at the head portion of the cylinder. A ratchet 312 is provided with in the exterior middle of the large needle seat 31 to engage with the sheath 5. The inner hole of the large needle seat 31 is formed of an upper hole section 313, an intermediate hole section 314 and a lower hole section 315 with their hole diameters successively increasing from the top to the bottom. The upper hole section 313 and the intermediate hole section 314 defining an upper flange 316 therebetween. The intermediate hole section 314 and the lower hole section 315 defining a lower flange 317 therebetween. The top of the cylinder 1 abuts against the bottom of the lower flange 317. The small needle seat 32 is retractable inside the plunger 2. The upper section of the small needle seat 32 is fitted within the inner hole of the large needle seat 31 by virtue of static friction force, and the lower section of the small needle seat 32 is disposed within the mounting hole 11 of the head portion of the cylinder. In this embodiment, the profile of the small needle seat 32 is formed of an upper section 321, an intermediate section 322 and a lower section 323 with their diameters successively increasing. In the structure thereof as shown in FIGS. 1 and 4, the upper section 321 and the intermediate section 322 define an upper boss 324 therebetween, and the intermediate section 322 and the lower section 323 define a lower boss 325 therebetween. Hereby, the upper section 321 is located within the upper hole section 313 of the large needle seat, the intermediate section 322 is located within the intermediate hole section 314 of the large needle seat while being hermetically fitted therewith. The lower section 323 is fitted with the mounting hole 11 of the cylinder and, at the same time, the upper boss 324 abutting against the upper flange 316 and the lower boss 325 abutting against the lower flange 317. The front portion of the inner hole of the small needle seat 32 is a fine through hole 326 with a needle 4 mounted thereon. The rear portion of the inner hole of the small needle seat 32 is formed of a transition hole section 327 and a connection hole section 328 while the inner diameter of the transition hole section 327 is smaller than that of the connection hole section 328. The length of the connection hole section 328 is on the order of 1.5 to 2 mm, and the bottom thereof is provided with a snap-fit projection 3281.

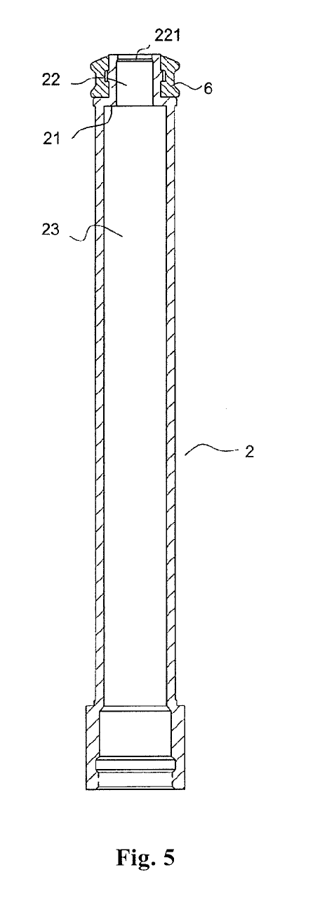

As shown in FIGS. 1 and 5, the plunger 2 is provided with a rubber plug 6 at the head portion thereof. The interior of the plunger 2 is a through hole. A boss surface 21 is provided within the through hole which divides the inner hole into a small hole diameter section 22 and a large hole diameter section 23. A friction flange 221 is provided in the opening portion of the small hole diameter section 22. The through hole is provided at the front portion thereof with an elastically reset device 7 capable of taking the small needle seat back into the through hole after injection. The through hole is hermetically engaged with a push handle 8 at the tail portion thereof.

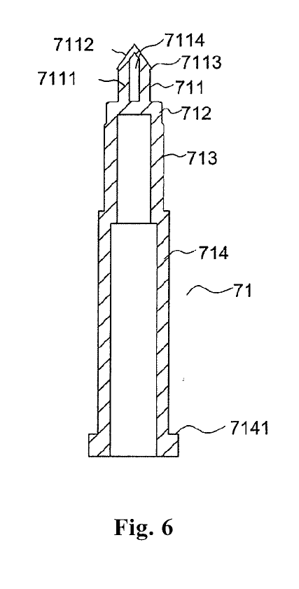

As shown in FIGS. 1 and 6, the elastically reset device 7 is comprised of a spring fixed seat 71 and a compression spring 72. The spring fixed seat 71 comprises a head portion 711 and an upper trunk portion 712 both protruding out of the through hole, and a lower trunk portion 713 and a foot portion 714 both positioned within the through hole. The head portion 711 of the spring fixed seat can enter the connection hole section 328 of the small needle seat 32 during injection. The top of the head portion 711 of the spring fixed seat is a flat square structure 7111, two sides of the flat square structure 7111 being conical bevels 7112 and the bottom of the conical bevel 7112 protruding outwardly to form a snap ring 7113. The maximum outer diameter of the snap ring 7113 is smaller than the inner diameter of the connection hole section 328 of the small needle seat but greater than the minimum inner diameter of the snap-fit projection 3281. The snap ring 7113 is able to engage with the snap-fit projection 3281 of the small needle seat when the spring fixed seat 71 is pulled down. Here, the flat square structure 7111 is provided with a through hole 7114 in order to enhance the deform performance of the head portion 711. The through hole in this embodiment is a rectangular hole. The upper trunk portion 712 has a length smaller than that of the connection hole section 328 of small needle seat and has an outer diameter smaller than the minimum inner diameter of the friction flange 221 but greater than the mounting hole 11 of the head portion of the cylinder. The lower trunk portion 713 is in clearance fit with the small hole diameter section 22 of the through hole while in tight fit with the friction flange 221 on the small hole diameter section 22. The top end face of the foot portion 714 abuts against the boss surface 21 of the through hole. The foot portion 714 protruding outwardly at the bottom thereof to form a block 7141. The compression spring 72 being mounted between the block 7141 and the boss surface 21 of the through hole. The restoring force of the compression spring 72 is, before the compression spring is triggered, smaller than the friction generated by the tight fit between the lower trunk portion 713 of the spring fixed seat and the friction flange 221. And the restoring force can, after the compression spring is triggered, draw the small needle seat 32 with the needle 4 back into the plunger 2.

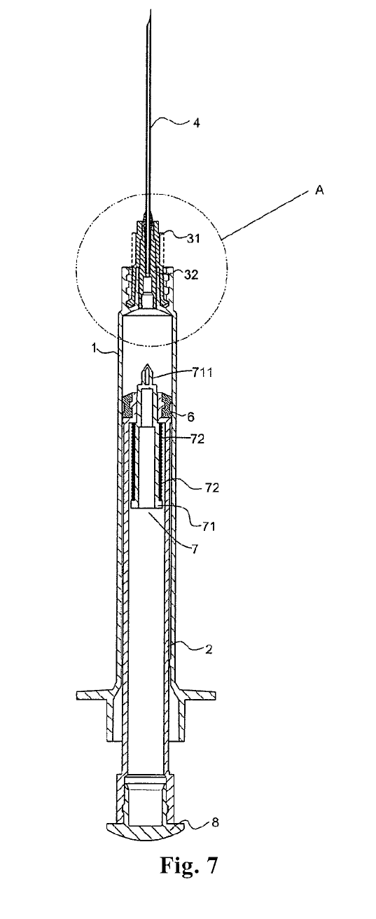

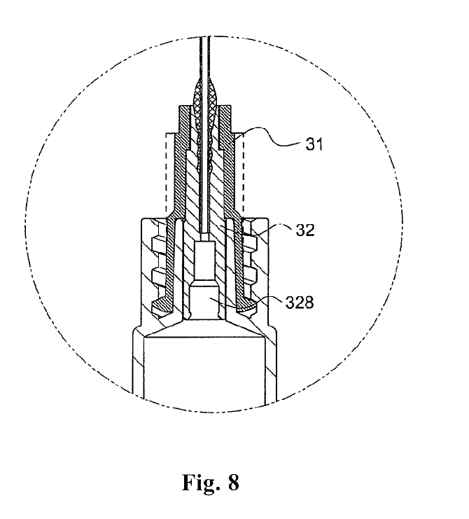

As shown in FIGS. 7 and 8 which are structural drawings of the present invention before injection. At this moment, the flat square structure 7111 of the spring fixed seat with a conical surface does not deeply go into the connection hole section 328 of the small needle seat. At this moment, the elastically reset device 7 is not actuated or triggered with the spring 72 being in a compressed state. The injection liquid flows toward the needle 4 from the inner hole of the small needle seat 32 as the plunger 2 is continuously pushed in.

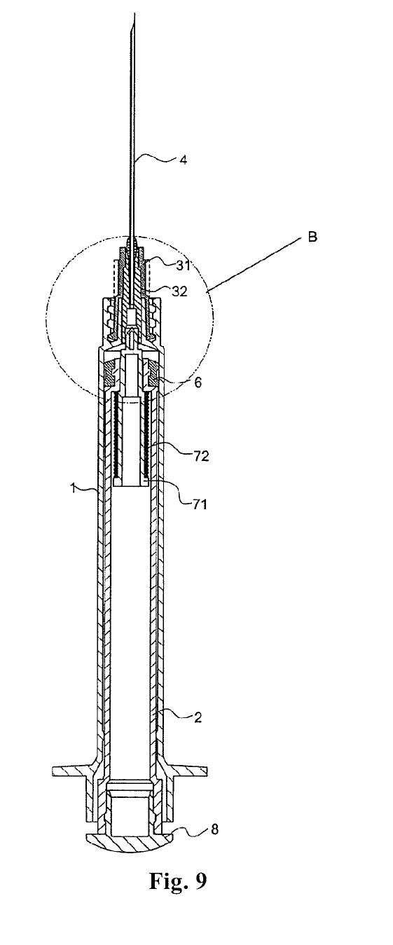

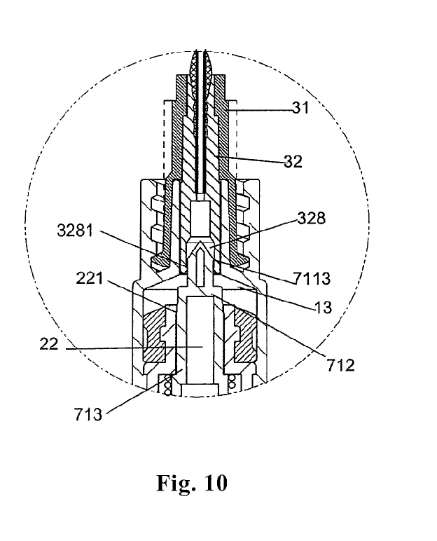

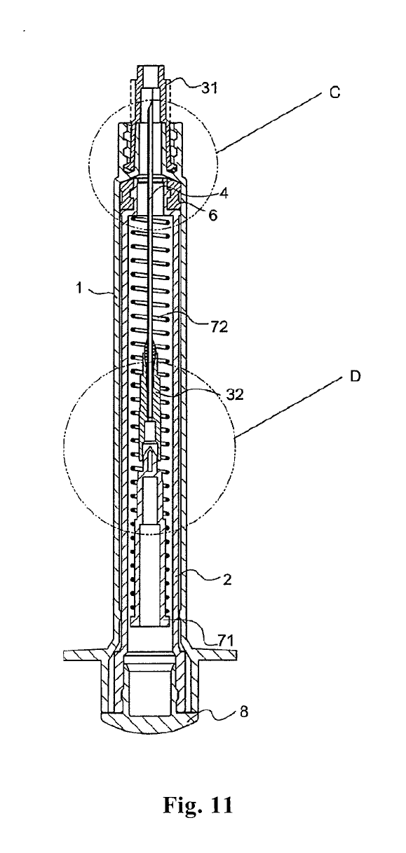

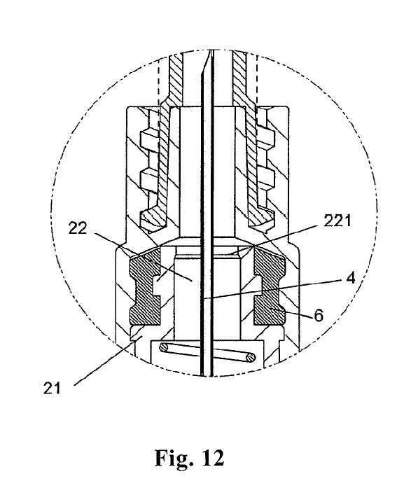

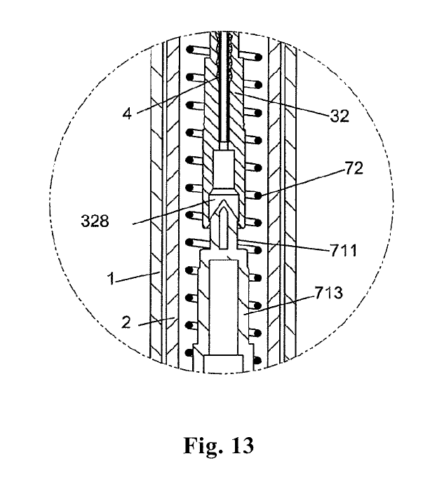

As shown in FIGS. 9 and 10 which are structural drawings of the elastically reset device 7 of the present invention before triggered while during injection. At this moment, the snap ring 7113 on the flat square structure 7111 of the spring fixed seat 71 head portion is deformed by virtue of a forward force until the snap ring 7113 gets across the snap-fit projection 3281 and enters into the connection hole section 328. Then the deformation is restored. The spring reset device 7 at this moment is not actuated yet and the spring 72 is still in a compressed state. The injection liquid flows toward the needle 4 from the inner hole of the small needle seat 32 as the plunger 2 is continuously pushed in. Subsequently, the top end face of the upper trunk portion 712 of the spring fixed seat touches the transition bevel 13 of the inner hole of the cylinder. At this moment, the spring fixed seat 71 is caught so as not to move forward. As the plunger 2 moves forward further, during the rubber plug 6 is quickly pushed to the bottom, the plunger 2 will move with respect to the spring fixed seat 71. The friction flange 221 on the small hole diameter section 22 of the inner hole of the plunger disengages from the lower trunk portion 713 of the spring fixed seat that is in tight fit therewith and enters into the upper trunk portion 712. This causes the friction therebetween disappears instantly. At this moment, the spring fixed seat 71 quickly springs back under action of the restoring force of the reset spring 72. At the same time, the snap ring 7113 engages with the snap-fit projection 3281 of the small needle seat to take the small needle seat 3281 and the needle 4 to enter into the through hole of the plunger 2. The result is as shown in FIGS. 11, 12 and 13. After injection, the injection needle 4 will not protrude out of the front end face of the cylinder 1. Thus it avoids accidental injury and thoroughly eliminating risk to the operator during recovery of syringes. Besides, it ensures extreme little medicament residual after injection is accomplished, thus avoiding to the personnel pollution caused by the residual medicament liquor.

In addition, the match structure between the small needle seat 32 and the large needle seat 31 can completely satisfy the stability requirements to the small needle seat 32 during injection. As another embodiment, in order to further ensure a better stability of the small needle seat 32 during injection and therefore to relieve the pain feeling of the patient, a stop structure can be additionally provided between the intermediate section 322 of the small needle seat and the intermediate hole section 314 of the inner hole of the large needle seat to prevent the small needle seat 32 from rotating. As shown in FIG. 3, this stop structure comprises a lug 3221 provided on the intermediate section 322 of the small needle seat and a depression 3141 provided on the intermediate hole section 314 of the large needle seat. The engagement connection between the lug 3221 and depression 3141 is a tight fit. Of course, the present invention can also use more lugs 3221 and more depressions 3141 engaging with each other to ensure that the lugs 3221 would not rotate.

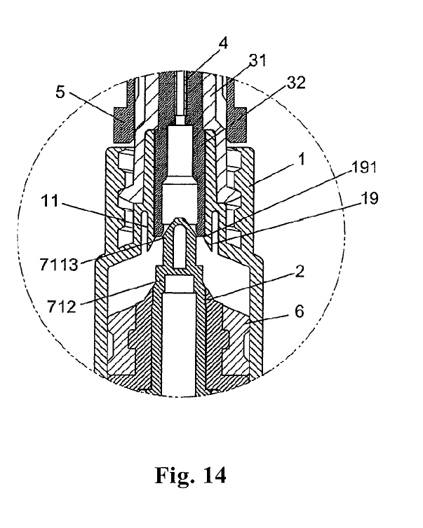

FIG. 14 shows another embodiment according to the present invention. It is different from the foregoing embodiment in that a stop structure of small needle seat is provided at the bottom of the mounting hole 11 of the cylinder which can be pushed off under the action of the head portion of the plunger 2. In this embodiment, the stop structure of the small needle seat is an elastic brace claw 19. A barb 191 is provided at the internal lower portion of the elastic brace claw 19 to engage with the bottom of the small needle seat 32. A bevel structure is provided at the bottom of the elastic brace claw 19 with an inclination matching with the head portion of the bevel of the plunger 2. The bevel structure is inwardly displaced by a distance with respect to the head portion of the bevel of the plunger 2.

The stop structure is arranged that, when the plunger 2 is not fully pressed to the bottom of the cylinder 1, the barb 191 of the elastic brace claw 19 will engage the bottom of the small needle seat 32 to stop the small needle seat 32 which will ensure that during injection, the small needle seat 32 will not disengage from the large needle seat 31 to inwardly retract due to excessive counterforce. After the injection is accomplished while the plunger is pressed to the bottom, because the inclination of the bevel structure of the elastic brace claw 19 matches with the head portion of the bevel of the plunger 2 and the bevel structure is inwardly displaced by a distance with respect to the head portion of the bevel of the plunger 2, the head portion of the bevel of the plunger 2 will force the bottom of the elastic brace claw 19 to expand outward and to force, under further compression by the plunger 2, the elastic brace claw 19 to spread outward, such that the engagement connection between the barb 191 and the bottom of the small needle seat 32 is released, thus ensuring a normal retraction of the small needle seat 32.

In summary, the present invention provides a disposable safety syringe with an automatically retractable needle, which is reasonable and simple in structure, easy in processing, low in cost and superior and reliable in performance.

The above describes some preferred embodiments of the present invention. However, it is not intended to or not construed to limit this invention. It should be appreciated that a person skilled in the prior art can, without depart from the range of the technical solutions of this invention, use the technical contents disclosed above to make a modification or amendment thereto to achieve any equivalent embodiment. Any content made without depart from the range of the technical solutions of this invention, and any simple amendment, any equivalent replacement, or any modification made, substantially under this invention, to the above embodiments, fall within the range of the technical solutions of this invention.

1. A disposable safety syringe with an automatically retractable needle, comprising a cylinder, a plunger and a needle seat, wherein:
the needle seat is comprised of a large needle seat and a small needle seat, in which the large needle seat is connected with the cylinder;
the small needle seat is disposed within the large needle seat for mounting the needle;
the interior of the plunger is a through hole and an elastically reset device is provided in the front portion of the through hole for taking the small needle seat back into the through hole of the plunger after injection,
wherein the elastically reset device is comprised of a spring fixed seat and a compressed compression spring, in which under the action of the compression spring, the spring fixed seat forms with the through hole of the plunger a movable fit that has a temporary restraint.

2. The disposable safety syringe according to claim 1, wherein the small needle seat is fitted within the large needle seat by virtue of a static friction force.

3. The disposable safety syringe according to claim 2, wherein the small needle seat is provided with an inner hole in the center thereof and the rear portion of the inner hole is a connection hole section, a snap-fit projection being provided at the bottom of the connection hole section to cooperate with the elastically reset device for position restriction.

4. (canceled)

5. The disposable safety syringe according to claim 1, wherein the spring fixed seat comprises a head portion and an upper trunk portion protruding out of the through hole of the plunger, and a lower trunk portion within the through hole of the plunger, the head portion being provided with a connection structure to be fitted with the small needle seat, the upper trunk portion has an outer diameter smaller than that of the lower trunk portion so that when a friction flange disposed within the through hole of the plunger moves from the lower trunk portion to the upper trunk portion, the spring fixed seat is free from restraint and thereby retract to the bottom of the through hole of the plunger together with the small needle seat under the action of the spring.

6. The disposable safety syringe according to claim 5, wherein the top of the head portion of the spring fixed seat has a flat square structure, two sides of the flat square structure being conical bevels with a bottom thereof protruding outward to form a snap ring, the maximum outer diameter of the snap ring being smaller than the inner diameter of the connection hole section of the small needle seat but greater than the minimum inner diameter of the snap-fit projection, and the snap ring may engage with the snap-fit projection of the small needle seat for position restriction when the spring fixed seat is pulled down.

7. The disposable safety syringe according to claim 5, wherein a boss surface is provided in the through hole of the plunger to divide the inner hole into a small hole diameter section and a large hole diameter section, and an opening portion of the small hole diameter section is provided with the friction flange.

8. The disposable safety syringe according to claim 7, wherein the top of the head portion of the spring fixed seat has a flat square structure, two sides of the flat square structure being conical bevels with a bottom thereof protruding outward to form a snap ring, the maximum outer diameter of the snap ring being smaller than the inner diameter of the connection hole section of the small needle seat but greater than the minimum inner diameter of the snap-fit projection, and the snap ring may engage with the snap-fit projection of the small needle seat for limiting when the spring fixed seat is pulled down.

9. The disposable safety syringe according to claim 7, wherein the upper trunk portion of the spring fixed seat has a length smaller than that of the connection hole section of the small needle seat and has an outer diameter smaller than the minimum inner diameter of the friction flange but greater than an mounting hole of the head portion of the cylinder; the lower trunk portion is in clearance fit with the smaller hole diameter section of the through hole of the plunger while in tight fit with the friction flange of the through hole of the plunger; the top end face of the foot portion of the spring fixed seat abuts against the boss surface of the through hole of the plunger, the foot portion of the spring fixed seat protruding outward at its bottom to form a block, the compression spring being mounted between the block and the boss surface of the through hole, the restoring force of the compression spring is smaller than the friction generated by the tight fit between the lower trunk portion of the spring fixed seat and the frictional flange when the compression spring is in a compressed state and before it is not triggered, and the restoring force can draw the small needle seat and the needle back into the plunger after the compression spring is triggered.

10. The disposable safety syringe according to claim 8, wherein a through hole is provided on the flat square structure of the head portion of the spring fixed seat for increasing deformation.

11. The disposable safety syringe according to claim 1, wherein the large needle seat is in threaded connection with the cylinder.

12. The disposable safety syringe according to claim 11, wherein the external bottom of the large needle seat is provided with a convex ring and the head portion of the cylinder is provided with a recess in which threads are provided to connect with the convex ring.

13. The disposable safety syringe according to claim 1, wherein the external middle of the large needle seat is provided with a ratchet to engage with a sheath.

14. The disposable safety syringe according to claim 1, wherein the inner hole of the large needle seat is comprised of an upper hole section, an intermediate hole section and a lower hole section with their hole diameters successively increasing from the top to the bottom, the upper hole section and the intermediate hole section defining an upper flange therebetween, and the intermediate hole section and the lower hole section defining a lower flange therebetween; the small needle seat is comprised of an upper section, an intermediate section and a lower section with their hole diameters successively increasing, the upper section and the intermediate section defining an upper boss therebetween, and the intermediate section and the lower section defining a lower boss therebetween; the upper section is located within the upper hole section of the large needle seat, the intermediate section being located within the intermediate hole section of the large needle seat and being hermetically fit therewith, and the lower section being fitted with the mounting hole of the cylinder; and the top of the cylinder and the upper boss of the small needle seat abut against the upper flange of the large needle seat, and the lower boss of the small needle seat abutting against the lower flange of the large needle seat.

15. The disposable safety syringe according to claim 14, wherein a stop structure is provided between the intermediate section of the small needle seat and the intermediate hole section of the inner hole of the large needle seat to prevent the small needle seat from rotating, which comprises a lug disposed on the intermediate section of the small needle seat and a depression disposed on the intermediate hole section of the large needle seat.

16. The disposable safety syringe according to claim 3, wherein the length of the connection hole section of the inner hole of the small needle seat is on the order of 1.5 to 2 mm.

17. The disposable safety syringe according to claim 1, wherein the inner hole of the cylinder is provided with a snap ring at the bottom thereof.

18. The disposable safety syringe according to claim 1, wherein the cylinder is provided with a mounting hole at the front portion thereof, the mounting hole being in connection with the inner hole of the cylinder by a transition bevel.

19. The disposable safety syringe according to claim 18, wherein a stop structure of the small needle seat is provided at the bottom of the mounting hole which can be pushed off under the action of the head portion of the plunger.

20. The disposable safety syringe according to claim 19, wherein the stop structure of the small needle seat is an elastic brace claw, a barb being provided in the internal lower portion of the elastic brace claw to engage with the bottom of the small needle seat, a bevel structure being provided at the bottom of the elastic brace claw with an inclination matching with the head portion of the bevel of the plunger, and the bevel structure being inward displaced by a distance with respect to the head portion of the bevel of the plunger.
